(19) United States
(12) Patent Application Publication
(10) Pub. No.: US 2014/0249420 A1
(43) Pub. Date: Sep. 4, 2014

(54) ULTRASOUND DIAGNOSTIC APPARATUS

(71) Applicant: KONICA MINOLTA, INC., Tokyo (JP)

(72) Inventors: Mutsuhiro AKAHANE, Tokyo (JP); Kazuya OSADA, Tokyo (JP)

(73) Assignee: KONICA MINOLTA, INC., Tokyo (JP)

(21) Appl. No.: 14/194,387

(22) Filed: Feb. 28, 2014

(30) Foreign Application Priority Data
Mar. 4, 2013 (JP) 2013-041379

(51) Int. Cl. A61B 8/00

(52) U.S. Cl. CPC A61B 8/54
USPC 600/459; 600/437

(57) ABSTRACT

An ultrasound diagnostic apparatus includes a signal generation unit, amplitude adjustment units and a control unit. The signal generation unit generates a pulse signal with a predetermined voltage amplitude. The amplitude adjustment units individually change the voltage amplitude of the pulse signal so as to output pulse signals with individual voltage amplitudes to their respective transducers. The control unit controls change amounts of the voltage amplitude made by the amplitude adjustment units. Each amplitude adjustment unit includes resistive elements and switching units which change a conductive state of their respective resistive elements. The control unit outputs a control signal to set a combination of ON and/or OFF of the switching units to change a load of the amplitude adjustment unit so as to apply to the transducer a voltage for an amplitude of ultrasound which is transmitted from each of the transducers.

### 1. FIELD OF THE INVENTION

The present invention relates to an ultrasound diagnostic apparatus.

### 2. DESCRIPTION OF THE RELATED ART

There has been a conventional ultrasound diagnostic apparatus which generates ultrasound and detects its reflected waves to provide medical information. Ultrasound diagnosis is nondestructive and noninvasive and hence is used to examine inside of living bodies.

In the ultrasound diagnostic apparatus, an ultrasound probe to transmit ultrasound and receive and detect its reflected waves is used. The ultrasound probe has a predetermined number of transducers arranged. The transducers deform when voltage pulses are applied thereto according to the voltages so as to generate (transmit) ultrasound of a desired frequency, and vibrate when receiving its reflected waves of the frequency band and convert the vibrations into voltage signals. By appropriately selecting transducers to which voltages are applied and controlling timings to apply the voltages to the selected transducers, ultrasound having directivity in a desired direction and being focused at a desired distance can be generated.

However, the transducers generate, in addition to the ultrasound (main lobes) in a desired direction, complementary waves (side lobes) having directivity in a direction oblique to the desired direction. This causes a problem that the transducers detect reflected waves of the side lobes and accordingly a virtual image (artifact) is formed at a false position. Then, there has been a conventional art to individually control the voltages applied to the transducers so as to vary amplitudes of ultrasound to be transmitted from the transducers, thereby reducing the side lobes.

There is described in Japanese Patent Application Laid-Open. Publication No. H09-234202 an art to prepare a plurality of voltages of power sources and switch the power sources to be connected to each transducer for an applied voltage. Further, there is described in Japanese Patent Application Laid-Open Publication No. 2003-275203 an art to convert a standardized digital waveform signal into an analog waveform signal and then attenuate the amplitude of the analog waveform signal to a desired level with an attenuator circuit. Further, there is described in Japanese Patent Application Laid-Open Publication No. 2008-68014 an art to stop rise of an applied voltage with a comparator when the applied voltage reaches a desired voltage during rise of an applied voltage pulse so as to apply a desired voltage to a transducer. Further, there is described in Japanese Patent Application Laid-Open Publication No. H07-155322 an art to change a duty cycle of a pulse voltage applied to a transducer and appropriately control its timing so as to apply an effective voltage for a desired applied voltage to the transducer.

### BRIEF SUMMARY OF THE INVENTION

However, ultrasound diagnostic apparatuses configured by the above-described conventional arts each require a plurality of circuits/devices which are large in size, such as a power supply circuit and a D/A convertor, and/or require high accuracy control, such as accurate timing control within a very short time. Hence, it is difficult to easily manufacture the ultrasound diagnostic apparatuses at low cost.

Objects of the present invention include providing an ultrasound diagnostic apparatus having a function to individually control applied voltages to transducers, the ultrasound diagnostic apparatus being easily manufactured at low cost.

In order to achieve at least one of the objects, according to an aspect of the present invention, there is provided an ultrasound diagnostic apparatus which outputs a pulse signal to each of a plurality of transducers to make each of the transducers transmit ultrasound and obtains a reception signal based on ultrasound received by each of the transducers, the ultrasound diagnostic apparatus including: a signal generation unit which generates the pulse signal with a predetermined voltage amplitude; a plurality of amplitude adjustment units which individually change the voltage amplitude of the pulse signal so as to output pulse signals with individual voltage amplitudes to the respective transducers; and a control unit which controls change amounts of the voltage amplitude made by the amplitude adjustment units, wherein each of the amplitude adjustment units includes a plurality of resistive elements and a plurality of switching units which change a conductive state of the respective resistive elements, the control unit outputs a control signal to set a combination of ON and/or OFF of the switching units to change a load of the amplitude adjustment unit so as to apply a voltage for an amplitude of the ultrasound, which is transmitted from each of the transducers, to the transducer.

Preferably, in the ultrasound diagnostic apparatus, the resistive elements in the amplitude adjustment unit are electrically connected to the transducer in parallel.

Preferably, the ultrasound diagnostic apparatus further includes an ultrasound probe including the transducers and a connection unit to be connected with a main body of the ultrasound diagnostic apparatus, wherein the amplitude adjustment units are disposed in the ultrasound probe.

Preferably, the ultrasound diagnostic apparatus further includes a signal processing unit which processes the reception signal, wherein with respect to each of the amplitude adjustment units and each of the transducers, a reception line to convey the reception signal to the signal processing unit and a transmission line to convey the pulse signal from the signal generation unit are connected at a predetermined node to a common line connected to the transducer, the amplitude adjustment unit is disposed on the transmission line, and between the node and the amplitude adjustment unit, a selective passing unit which selectively allows the pulse signal to pass is disposed.

Preferably, in the ultrasound diagnostic apparatus, the switching units each are an analog switch or a mechanical relay switch.

Preferably, in the ultrasound diagnostic apparatus, resistance values of the resistive elements in the amplitude adjustment unit are all different from each other.

### BRIEF DESCRIPTION OF THE DRAWINGS

The present invention is fully understood from the detailed description given hereinafter and the accompanying drawings, which are given by way of illustration only and thus are not intended to limit the present invention, wherein:

FIG. 1 is an overall view of an ultrasound diagnostic apparatus according to a first embodiment of the present invention;

FIG. 2 is a block diagram showing the internal configuration of the ultrasound diagnostic apparatus according to the first embodiment;

FIG. 3 shows a circuit configuration relating to transmission/reception of signals to/from an ultrasound probe in the ultrasound diagnostic apparatus according to the first embodiment;

FIG. 4 is a block diagram showing the internal configuration of an ultrasound diagnostic apparatus according to a second embodiment of the present invention;

FIG. 5 shows a circuit configuration relating to transmission/reception of signals to/from an ultrasound probe in the ultrasound diagnostic apparatus according to the second embodiment;

FIG. 6 shows a circuit configuration relating to transmission/reception of signals to/from an ultrasound probe in an ultrasound diagnostic apparatus according to a third embodiment of the present invention;

FIG. 7 shows a circuit configuration relating to transmission/reception of signals to/from an ultrasound probe in an ultrasound diagnostic apparatus according to a fourth embodiment of the present invention;

FIG. 8 is a block diagram showing the internal configuration of an ultrasound diagnostic apparatus according to each of a fifth embodiment and a sixth embodiment of the present invention;

FIG. 9A shows a circuit configuration relating to transmission/reception of signals to/from an ultrasound probe in an ultrasound diagnostic apparatus according to the fifth embodiment;

FIG. 9B shows a circuit configuration relating to transmission/reception of signals to/from an ultrasound probe in an ultrasound diagnostic apparatus according to the sixth embodiment;

FIG. 10A shows a circuit configuration relating to transmission/reception of signals to/from an ultrasound probe in an ultrasound diagnostic apparatus according to a seventh embodiment of the present invention;

FIG. 10B shows a circuit configuration relating to transmission/reception of signals to/from an ultrasound probe in an ultrasound diagnostic apparatus according to an eighth embodiment of the present invention;

FIG. 11A shows a circuit configuration relating to transmission/reception of signals to/from an ultrasound probe in an ultrasound diagnostic apparatus according to a ninth embodiment of the present invention;

FIG. 11B shows a circuit configuration relating to transmission/reception of signals to/from an ultrasound probe in an ultrasound diagnostic apparatus according to a tenth embodiment of the present invention; and

FIG. 12 shows a circuit configuration relating to transmission/reception of signals to/from an ultrasound probe in an ultrasound diagnostic apparatus according to an eleventh embodiment of the present invention.

### DETAILED DESCRIPTION OF THE INVENTION

In the following, embodiments of the present invention are described with reference to the drawings.

### First Embodiment

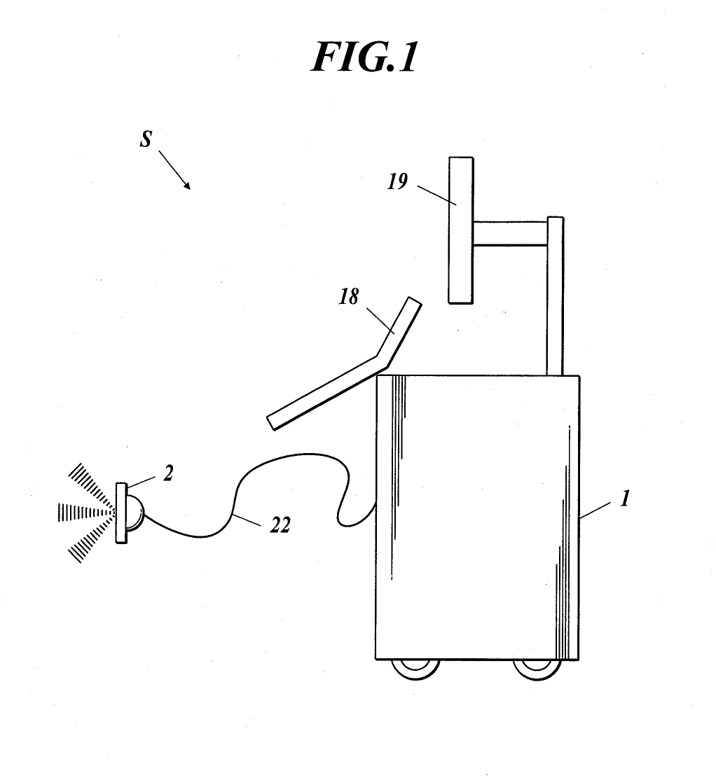

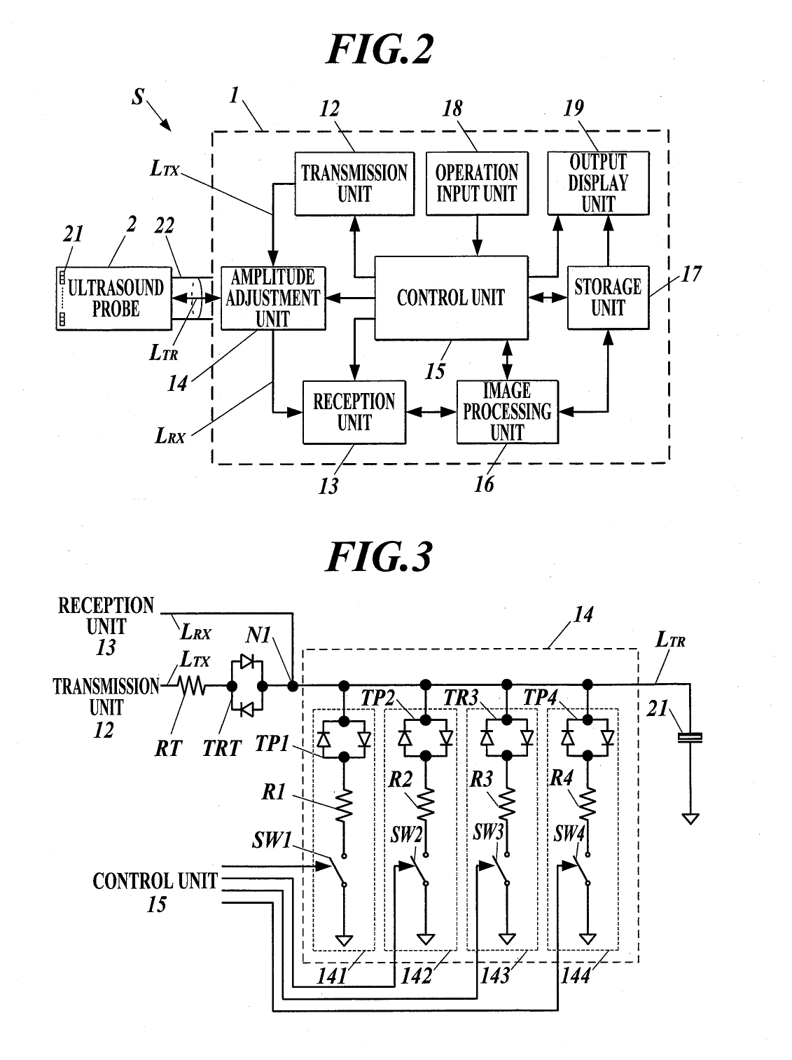

FIG. 1 is an overall view of an ultrasound diagnostic apparatus S according to a first embodiment of the present invention. FIG. 2 is a block diagram showing the internal configuration of the ultrasound diagnostic apparatus S according to the first embodiment. In the following, the ultrasound diagnostic apparatus S of the first embodiment is described with reference to FIGS. 1 and 2.

The ultrasound diagnostic apparatus S of the first embodiment includes a main body 1 of the ultrasound diagnostic apparatus S and an ultrasound probe 2.

The ultrasound probe 2 transmits ultrasound to a not-shown subject such as a living body and receives reflected waves (echoes) generated by the transmitted ultrasound being reflected by the subject. The ultrasound probe 2 includes a plurality of transducers 21 and a cable 22 (connection unit). The cable 22 has a connector (not shown) at one end to be connected with the main body 1. The ultrasound probe 2 is connected to the main body 1 through the cable 22 so as to be used.

The transducers 21 are composed of piezoelectric elements and arranged in a one-dimensional array in an orientation direction. When voltage pulses (pulse signals) are applied to the transducers 21, the transducers 21 deform, and ultrasound is transmitted with amplitudes corresponding to the voltages. Further, when ultrasound of a predetermined frequency band is conveyed to the transducers 21, the thicknesses of the transducers 21 vary (the transducers 21 vibrate), so that electrical signals (reception signals) corresponding to the variations (vibrations) are generated.

In the first embodiment, the ultrasound probe 2 has 192 transducers 21, for example. The number of transducers 21 can be set appropriately. The transducers 21 may be arranged in a two-dimensional array. The ultrasound probe 2 adopts electronic scanning or mechanical scanning and adopts, for the scanning, linear scanning, sector scanning or convex scanning. Further, the bandwidth of the reception signals in the ultrasound probe 2 can be set appropriately.

The ultrasound diagnostic apparatus S is configured in such a way that one of different types of ultrasound probes 2 is connected to the main body 1 depending on a diagnosis target (a part of a living body, for example).

The main body 1 includes, for example, a transmission unit 12 (signal generation unit), a reception unit 13 (signal processing unit), amplitude adjustment units 14, a control unit 15, an image processing unit 16, a storage unit 17, an operation input unit 18 and an output display unit 19.

The transmission unit 12 outputs pulse signals supplied to the ultrasound probe 2 in response to control signals input from the control unit 15 to make the ultrasound probe 2 generate ultrasound. The transmission unit 12 includes, for example, a clock generation circuit, a pulse generation circuit, a pulse width setting unit and a delay circuit. The clock generation circuit is a circuit to generate clock signals which determine transmission timings of the pulse signals and the transmission frequency thereof. The pulse generation circuit is a circuit to generate the bipolar rectangular pulses with a preset voltage amplitude at predetermined intervals. The pulse width setting unit sets the pulse width of the rectangular pulses output from the pulse generation circuit. The rectangular pulses generated at the pulse generation circuit are separated toward respective paths for the transducers 21 before or after the pulses are input to the pulse width setting unit. The delay circuit is a circuit to delay the transmission timings of the generated rectangular pulses by respective delay times which are individually set with respect to the respective paths for the transducers 21 to output the rectangular pulses to the respective transducers 21.

The reception unit 13 obtains, under control of the control unit 15, the reception signals input from the ultrasound probe 2. The reception unit 13 includes, for example, an amplifier, an A/D convertor circuit and a phasing addition circuit. The amplifier is a circuit to amplify the reception signals corresponding to ultrasound received by the transducers 21 of the ultrasound probe 2 at a preset amplification factor. The A/D convertor circuit is a circuit to convert the amplified reception signals into digital data at a predetermined sampling frequency. The phasing addition circuit is a circuit to give the delay times, which are individually set with respect to the respective paths for the transducers 21, to the A/D conversion-performed reception signals so as to phase the signals and to add up these signals (phasing addition) so as to generate sound ray data.

The amplitude adjustment units 14 are disposed on the respective paths for the transducers 21 and control the amplitude of the pulse signals, which are transmitted to the transducers 21 through the paths. The amplitude adjustment units 14 are described in detail below.

The control unit 15 includes a CPU (Central Processing Unit), an HDD (Hard Disk Drive) and a RAM (Random Access Memory). The CPU reads a program(s) of various programs stored in the HDD to load the program into the RAM and performs centralized control of actions of the components of the ultrasound diagnostic apparatus S in accordance with the loaded program. The HDD stores therein: a control program and various processing programs to make the ultrasound diagnostic apparatus S act; various setting data; and the like. These programs and setting data may be stored in, other than the HDD, an auxiliary storage device using a nonvolatile memory such as a flush memory in such a way as to be readable, writable and updatable. The RAM is a volatile memory such as an SRAM or a DRAM, provides a working memory space for the CPU, and stores therein temporary data.

The image processing unit 16 performs, independently from the CPU of the control unit 15, arithmetic processing to generate diagnostic images based on received ultrasound data. Examples of the diagnostic images include image data to be displayed on the output display unit 19 in near real-time, moving image data composed of a series of the image data, and still image data of snapshots. This arithmetic processing may be performed by the CPU of the control unit 15.

The storage unit 17 is, for example, a volatile memory such as a DRAM (Dynamic Random Access Memory). The storage unit 17 may be a nonvolatile memory of any type capable of high-speed rewriting or a combination of the nonvolatile memory and the volatile memory. The storage unit 17 stores therein the diagnostic image data for real-time display processed by the image processing unit 16 frame by frame. The diagnostic image data stored in the storage unit 17 is, under control of the control unit 15, read therefrom to be sent to the output display unit 19 or to be output to the outside of the ultrasound diagnostic apparatus S through a not-shown communication unit. If the output display unit 19 has a television system as its display system, a DSC (Digital Signal Converter) may be provided between the storage unit 17 and the output display unit 19 so that the image data is sent thereto after the scan format is converted.

The operation input unit 18 includes a press button switch, a keyboard, a mouse, a trackball or any combination thereof and converts user's input operations into operation signals to input the operation signals to the main body 1.

The output display unit 19 includes: a display screen using one of various display systems such as an LCD (Liquid Crystal Display), an organic EL (Electro-Luminescent) display, an inorganic EL display, a plasma display and a CRT (Cathode Ray Tube) display; and a driving unit therefor. The output display unit 19 generates drive signals for the display screen (display pixels) in response to control signals output from the CPU of the control unit 15 and in accordance with the image data generated by the image processing unit 16 and displays on the display screen a menu for ultrasound diagnosis, a status thereof and measurement data based on the received ultrasound.

The operation input unit 18 and/or the output display unit 19 may be integrated with a housing for the main body 1 or may be attached to the main body 1 through an USB cable(s). If the main body 1 is provided with an operation input terminal and a display output terminal, a peripheral for operation and a peripheral for display may be connected to the terminals so as to be used as the operation input unit 18 and the output display unit 19.

Next, amplitude control in the ultrasound diagnostic apparatus S of the first embodiment performed when ultrasound is transmitted is described.

FIG. 3 shows a circuit configuration relating to transmission/reception of signals to/from one transducer 21, namely, output of a pulse signal to one transducer 21 from the transmission unit 12 and input of a reception signal from the transducer 21 to the reception unit 13, according to the first embodiment.

The pulse signal output from the transmission unit 12 passes through a transmission line L_{TX} provided with internal resistance RT of a pulser (a circuit which generates pulse signals) and a T/R (Transmitter/Receiver) switch TRT (selective passing unit) to enter a common line L_{TR} for both transmission and reception of signals, namely, for both the pulse signal and the reception signal, from a node N1 to be input to an amplitude adjustment unit 14 and then transmitted to the transducer 21. On the other hand, the reception signal into which ultrasound received by the transducer 21 is converted passes through the amplitude adjustment unit 14, which is disposed on the common line L_{TR}, and then flows to a reception line L_{RX} from the node N1 to be output to the reception unit 13.

The T/R switch TRT includes two diodes disposed in antiparallel. The T/R switch TRT acts to allow the pulse signal with a large voltage amplitude transmitted from the transmission unit 12 to pass toward the common line L_{TR} while acting not to allow the reception signal with a small voltage amplitude transmitted from the transducer 21 to pass toward the transmission line L_{TX} and the transmission unit 12.

The ultrasound diagnostic apparatus S usually includes, in addition to the T/R switch TRT, a T/R switch (not shown) on the reception line L_{RX}, which extends from the node N1 to the reception unit 13. The T/R switch acts to allow the reception signal with a small voltage amplitude to pass toward the reception unit 13 while acting not to allow the pulse signal with a large voltage amplitude to pass toward the reception unit 13.

The amplitude adjustment unit 14 has a plurality of weighting units. In the first embodiment, the amplitude adjustment unit 14 has four weighting units 141 to 144. In the weighting unit 141, a diode switch TP1, a resistor R1 (resistor element) and a switch SW1 (switching unit) are connected in series in the order named. The weighing unit 141 is connected to the common line L_{TR} with one end of the diode switch TP1 and grounded with one end of the switch SW1. In the same manner, in the weighting unit 142, a diode switch TP2, a resistor R2 and a switch SW2 are connected; in the weighting unit 143, a diode switch TP3, a resistor R3 and a switch SW3 are connected; and in the weighting unit 144, a diode switch TP4, a resistor R4 and a switch SW4 are connected. One end of each of the diode switches TP2 to TP4 is connected to the common line L_{TR}, and one end of each of the switches SW2 to SW4 is grounded. Consequently, the weighting units 141 to 144, namely, the resistors R1 to R4, are arranged in parallel with the transducer 21.

The diode switches TP1 to TP4 allow a voltage signal with a predetermined positive threshold voltage or more or a predetermined negative threshold voltage or less to pass. The threshold voltages are determined by the forward voltage characteristics of the diodes of the diode switches TP1 to TP4. In the ultrasound diagnostic apparatus S, the voltage amplitude (for example, around 100 V) of the pulse signal(s) is much larger than the absolute value of each of these threshold voltages, whereas the voltage amplitude (usually, in the mV-range) of the reception signal(s) is smaller than the absolute value of each of these threshold voltages. Therefore, these diode switches TP1 to TP4 selectively allow the pulse signal to pass and do not allow the reception signal to pass.

On/OFF of the switches SW1 to SW4 is controlled on the basis of control signals from the control unit 15. These switches SW1 to SW4 have high voltage resistance so as to resist a high voltage which is output thereto as the pulse signal. The switches SW1 to SW4 are, for example, analog switches such as FETs. If the switches SW1 to SW4 each are open, thereby being in an OFF state, the weighting units 141 to 144 each are in a floating state and no voltage is applied to any of the resistors R1 to R4. Consequently, the transducer 21 transmits, on the basis of the pulse signal output from the transmission unit 12, ultrasound with an amplitude corresponding to a partial voltage applied to the transducer 21 of a voltage applied to the internal resistance RT of the pulser and the transducer 21 in series.

On the other hand, if one or more of the switches SW1 to SW4 is closed, thereby being in an ON state, the weighting units 141, 142, 143 and/or 144 which is/are in the ON state and the transducer 21 are connected in parallel between the node N1 and the grounding end. Consequently, the voltage applied to the transducer 21 on the basis of the pulse signal decreases in accordance with the magnitude(s) (resistance value(s)) of the resistor(s) included in the weighting unit(s) connected to the transducer 21 in parallel, and accordingly the amplitude of ultrasound to be transmitted from the transducer 21 decreases.

That is, voltages applied to the respective transducers 21 are individually controlled in response to control signals transmitted from the control unit 15 to the respective amplitude adjustment units 14 disposed on the paths for the respective transducers 21, so that the amplitudes of ultrasound to be transmitted from the respective transducers 21 are adjusted to follow a desired profile thereof. The profile to be used may have a distribution of amplitudes the same as/similar to a publically-known distribution thereof generally used in ultrasound diagnostic apparatuses. ON/OFF of each of the weighting units 141 to 144 of the transducers 21 can be controlled in such a way that the amplitudes of ultrasound to be transmitted from the transducers 21 are weighted with Hanning window as a window function with respect to a transmission direction of the ultrasound, for example.

The resistance values of the resistors R1 to R4 in each amplitude adjustment unit 14 are set appropriately. When the resistance values of the resistors R1 to R4 in each amplitude adjustment unit 14 are set to be all different from each other, more levels of an applied voltage to a transducer 21 are settable with a small number of resistors.

As described above, the ultrasound diagnostic apparatus S of the first embodiment includes the amplitude adjustment units 14 which individually adjust the voltage amplitude of pulse signals output from the transmission unit 12 for their respective transducers 21, to which the amplitude adjustment units 14 output the pulse signals, and output pulse signals with the individual voltage amplitudes to the respective transducers 21. Each amplitude adjustment unit 14 includes the resistors R1 to R4 and the switches SW1 to SW4 which change the conductive state of the respective resistors R1 to R4. The control unit 15 controls ON/OFF of each of the switches SW1 to SW4 so as to change and apply to the transducer 21 a partial voltage for an amplitude of ultrasound to be transmitted from the transducer 21. Thus, the amplitudes of ultrasound to be output from the respective transducers 21 can be individually controlled easily with a simple configuration, namely, only with the resistive elements and the switches such as FETs.

Further, four weighting units 141 to 144, namely, the resistors R1 to R4, in each amplitude adjustment unit 14 are electrically connected to a transducer 21 in parallel, so that a partial voltage against the preset internal resistance RT of the pulser is applied to the amplitude adjustment unit 14 and the transducer 21. Consequently, even if the resistance of transducers 21 varies depending on ultrasound probes 2, namely, even if an ultrasound probe 2 having a specification (frequency, shape or the like) different from that of the currently-connected ultrasound probe 2 is connected, partial voltages applied to the transducers 21 are not much different from those applied to the transducers 21 of the currently-connected ultrasound probe 2.

Further, the weighting units 141 to 144 in each amplitude adjustment unit 14 include the diode switches TP1 to TP4, respectively. Each of the diode switches TP1 to TP4 is composed of two diodes disposed in antiparallel. The diode switches TP1 to TP4 allow a large voltage of the pulse signal to pass so as to adjust the amplitude thereof while not allowing a small voltage of the reception signal to pass. Consequently, with a simple configuration, the reception signals can be input to the reception unit 13 without being attenuated. Accordingly, high-speed switching control of the switches SW1 to SW4 performed at the time when transmission and reception of ultrasound are switched is unnecessary in order that the only pulse signals are allowed to pass through the weighting units 141 to 144 in the amplitude adjustment units 14.

Further, analog switches having high voltage resistance, such as FETs, are used as the switches SW1 to SW4. Consequently, high voltage resistance and a high-speed action are achievable with a small and simple configuration.

Further, when the resistance values of the resistors R1 to R4 in each amplitude adjustment unit are set to be all different from each other, more levels of combined resistance are settable with a small number of resistors, and the number of components and/or the number of lines for control signals can be reduced.

### Second Embodiment

Next, an ultrasound diagnostic apparatus according to a second embodiment of the present invention is described.

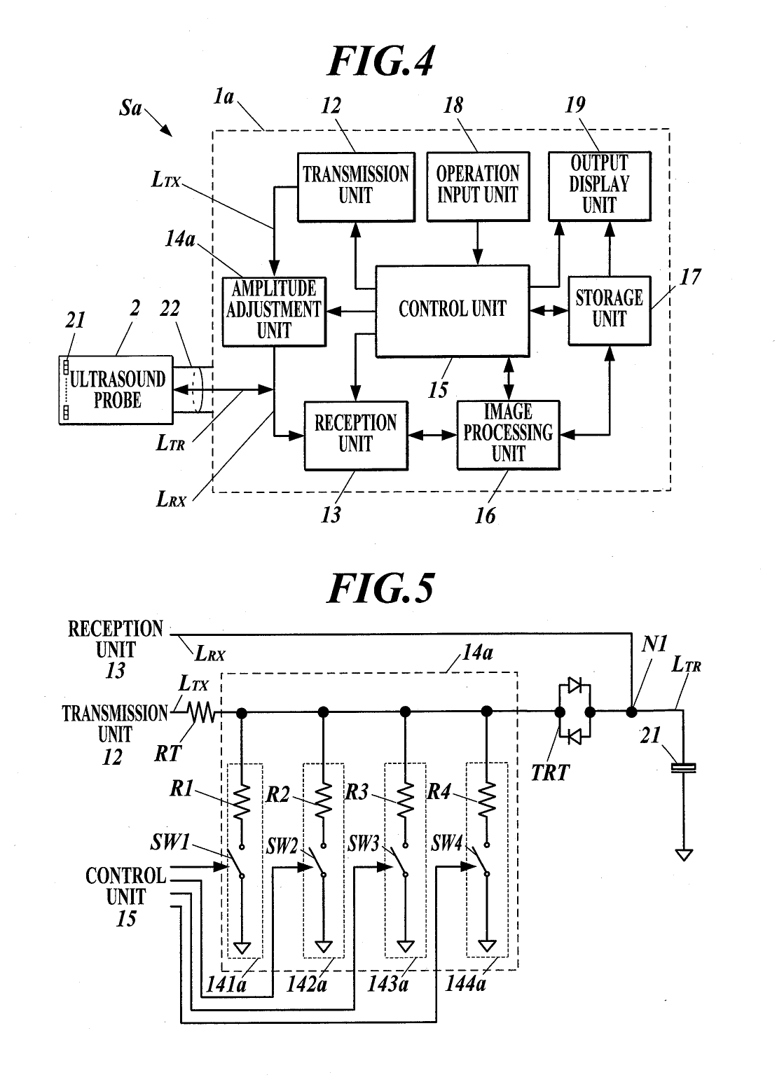

FIG. 4 is a block diagram showing the internal configuration of an ultrasound diagnostic apparatus Sa according to the second embodiment.

A main body 1a of the ultrasound diagnostic apparatus Sa of the second embodiment is the same as the main body 1 of the ultrasound diagnostic apparatus S of the first embodiment except that the arrangement and configuration of amplitude adjustment units 14a are different from those of the amplitude adjustment units 14. The same reference numbers are given to the same components, and description thereof is omitted herein.

In the main body 1a, pulse signals output from a transmission unit 12 are input to the amplitude adjustment units 14a, transmission lines L_{TX} which is threaded through the respective amplitude adjustment units 14a are tied with their respective reception lines L_{RX} which lead to a reception unit 13 so as to form common lines L_{TR}, and the main body 1a is connected with an ultrasound probe 2 through the common lines L_{TR}.

FIG. 5 shows a circuit configuration relating to transmission of a pulse signal and reception of a reception signal in the ultrasound diagnostic apparatus Sa of the second embodiment.

In the ultrasound diagnostic apparatus Sa of the second embodiment, as described above, an amplitude adjustment unit 14a is disposed on a transmission line L_{TX} between internal resistance RT of a pulser and a T/R switch TRT. In the amplitude adjustment unit 14a, weighting units 141a to 144a are connected in parallel. With this configuration, no reception signal is conveyed to the amplitude adjustment unit 14a owing to the T/R switch TRT, and hence none of diode switches TP1 to TP4 is provided in any of the weighting units 141a to 144a.

With the configuration of the ultrasound diagnostic apparatus Sa of the second embodiment too, the voltage amplitude of pulse signals for transducers 21 can be appropriately controlled, and ultrasound with desired amplitudes can be transmitted from the respective transducers 21 by setting the resistance values of resistors R1 to R4 and/or by controlling ON/OFF of each of switches SW1 to SW4 in the same manner as that in the ultrasound diagnostic apparatus S of the first embodiment.

Further, each amplitude adjustment unit 14a is disposed at a point of a transmission line L_{TX}, the point being apart from a reception line L_{RX} connected to the reception unit 13, and the T/R switch TRT is disposed on the transmission line L_{TX} to prevent backflow of a reception signal. Consequently, it is unnecessary to provide the amplitude adjustment unit 14a with a component or to make the amplitude adjustment unit 14a perform a switching action in order to prevent the reception signal from passing. Accordingly, the voltage amplitude of the pulse signals can be adjusted with a simpler configuration.

### Third Embodiment

Next, an ultrasound diagnostic apparatus according to a third embodiment of the present invention is described.

An ultrasound diagnostic apparatus S of the third embodiment is the same as the ultrasound diagnostic apparatus S of the first embodiment except that the amplitude adjustment units 14 are replaced by amplitude adjustment units 14b. The same reference numbers are given to the same components, and description thereof is omitted herein.

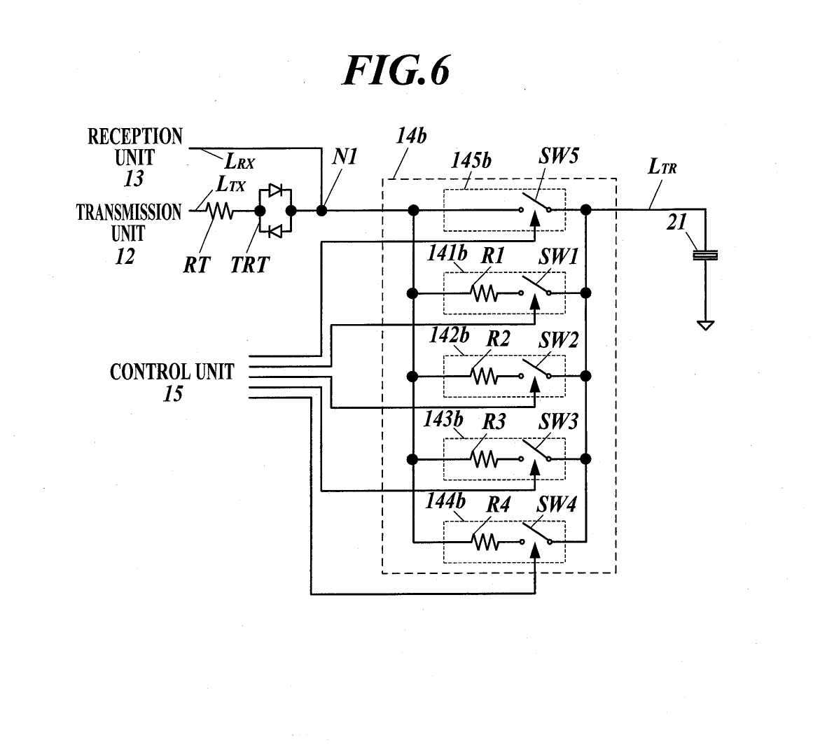

FIG. 6 shows a circuit configuration relating to transmission of a pulse signal and reception of a reception signal in the ultrasound diagnostic apparatus S of the third embodiment.

An amplitude adjustment unit 14b of the ultrasound diagnostic apparatus S of the third embodiment is disposed on a common line L_{TR} which extends from a node N1 to a transducer 21 so as to be connected to the transducer 21 in series. In the amplitude adjustment unit 14b, weighting units 141b to 144b are connected in parallel. In addition, a shortcut circuit 145b is connected to the weighting units 141b to 144b in parallel.

In the weighting unit 141b, a resistor R1 and a switch SW1 are connected in series. Similarly, in the weighting unit 142b, a resistor R2 and a switch SW2 are connected in series; in the weighting unit 143b, a resistor R3 and a switch SW3 are connected in series; and in the weighting unit 144b, a resistor R4 and a switch SW4 are connected in series.

The shortcut circuit 145b includes a switch SW5 only. On/Off of the switch SW5 is controlled on the basis of a control signal from a control unit 15, as with ON/OFF of each of the switches SW1 to SW4.

In a main body 1 of the ultrasound diagnostic apparatus S of the third embodiment, an output voltage from a transmission unit 12 is divided to be applied to internal resistance RT of a pulser, an amplitude adjustment unit 14 and a transducer 21. Then, ultrasound with an amplitude corresponding to a partial voltage applied to the transducer 21 is generated to be transmitted from the transducer 21. More specifically, when ultrasound is transmitted, the switch SW5 is made to be OFF, and ON/OFF of each of the switches SW1 to SW4 is appropriately controlled, so that a partial voltage applied to the amplitude adjustment unit 14b varies and accordingly the amplitude of ultrasound to be transmitted varies. On the other hand, when ultrasound is received, the switch SW5 is made to be ON in order that a reception signal bypasses the resistors R1 to R4, so that the reception signal based on the received ultrasound is transmitted to a reception unit 13 with no attenuation of the amplitude (strength) of the reception signal.

As described above, in the ultrasound diagnostic apparatus S of the third embodiment, the resistors R1 to R4 in each amplitude adjustment unit 14b are electrically connected to a transducer 21 in series. Consequently, as compared with a case in which the resistors R1 to R4 are electrically connected to the transducer 21 in parallel, the value of combined resistance is large, so that current is reduced as a whole, and accordingly power consumption can be reduced.

### Fourth Embodiment

Next, an ultrasound diagnostic apparatus according to a fourth embodiment of the present invention is described.

An ultrasound diagnostic apparatus Sa of the fourth embodiment is the same as the ultrasound diagnostic apparatus Sa of the second embodiment except that the amplitude adjustment units 14a are replaced by amplitude adjustment units 14c. The same reference numbers are given to the same components, and description thereof is omitted herein.

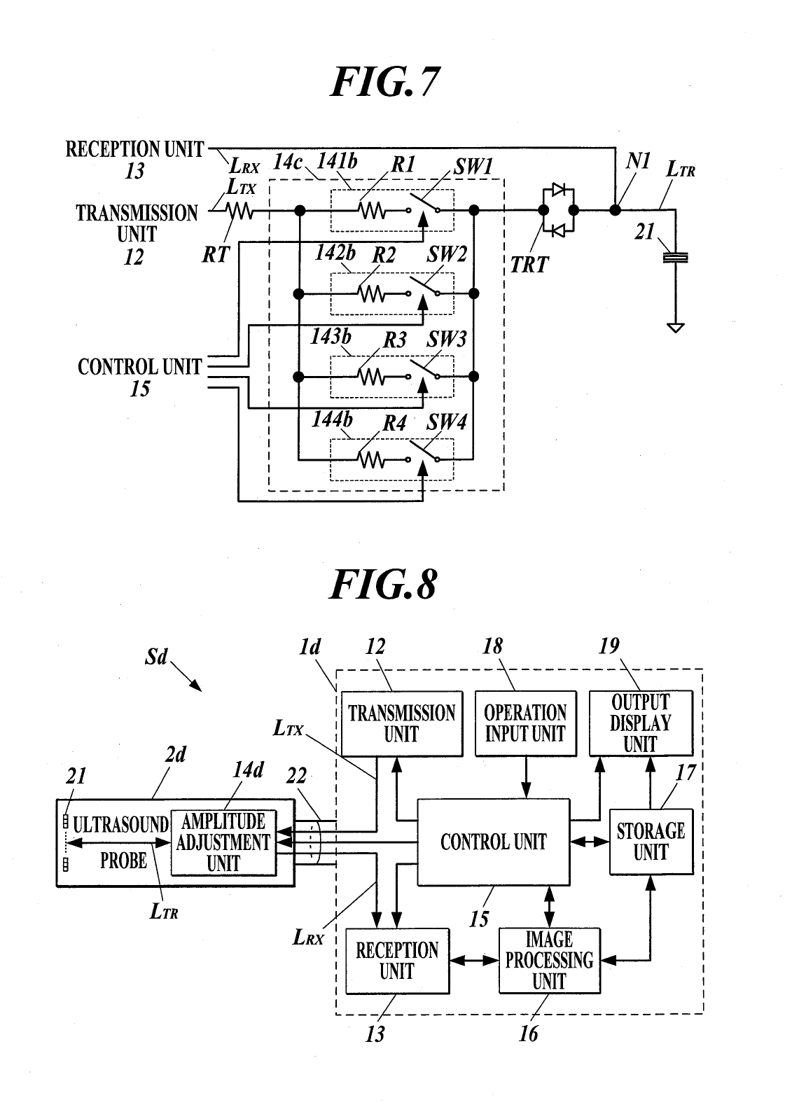

FIG. 7 shows a circuit configuration relating to transmission of a pulse signal and reception of a reception signal in the ultrasound diagnostic apparatus Sa of the fourth embodiment.

An amplitude adjustment unit 14c of a main body 1a of the ultrasound diagnostic apparatus Sa of the fourth embodiment is disposed on a transmission line L_{TX} between internal resistance RT of a pulser and a T/R switch TRT. In the amplitude adjustment unit 14c, weighting units 141b to 144b are connected in parallel. No reception signal flows to the amplitude adjustment unit 14c owing to the T/R switch TRT, and hence a shortcut circuit 145b to allow a reception signal to pass is not provided. However, a shortcut circuit 145b may be connected to the weighting units 141b to 144b in parallel. In this case, when a switch SW5 therein is ON, a voltage pulse as a pulse signal can be applied to a transducer 21 with no attenuation of the voltage amplitude of the pulse signal through resistors R1 to R4.

In the ultrasound diagnostic apparatus Sa of the fourth embodiment, as with the ultrasound diagnostic apparatus S of the third embodiment, a pulse signal with a certain voltage amplitude is divided to be applied to internal resistance RT of a pulser, one or more of the resistors R1 to R4 of an amplitude adjustment unit 14c and a transducer 21. Then, ultrasound with an amplitude corresponding to a partial voltage applied to the transducer 21 is generated to be transmitted. More specifically, when ultrasound is transmitted, (i) ON/OFF of each of switches SW1 to SW4 is controlled in such a way that a partial voltage applied to the amplitude adjustment unit 14c decreases, and in relation to it, a partial voltage applied to the transducer 21 increases, and accordingly the amplitude of ultrasound to be transmitted increases, or (ii) ON/OFF of each of the switches SW1 to SW4 is controlled in such a way that a partial voltage applied to the amplitude adjustment unit 14c increases, and in relation to it, a partial voltage applied to the transducer 21 decreases, and accordingly the amplitude of ultrasound to be transmitted decreases.

As described above, in the ultrasound diagnostic apparatus Sa of the fourth embodiment, each amplitude adjustment unit 14c (resistors R1 to R4) is disposed on a transmission line L_{TX} between internal resistance RT of a pulser and a T/R switch TRT to be electrically connected to the internal resistance RT of the pulser and the T/R switch TRT in series. In the amplitude adjustment unit 14c, the weighting units 141b to 144b are connected in parallel. In the ultrasound diagnostic apparatus Sa having this configuration, no reception signal flows to the amplitude adjustment units 14c, and the amplitudes of ultrasound to be transmitted from the transducers 21 can be individually controlled appropriately and easily with a simple configuration while unnecessary power consumption in the amplitude adjustment units 14c is prevented.

### Fifth Embodiment and Sixth Embodiment

Next, ultrasound diagnostic apparatuses according to a fifth embodiment and a sixth embodiment of the present invention are described.

FIG. 8 is a block diagram showing the internal configuration of each of ultrasound diagnostic apparatuses Sd according to the fifth embodiment and the sixth embodiment.

In the ultrasound diagnostic apparatus Sd, amplitude adjustment units 14d are disposed in an ultrasound probe 2d. Pulse signals transmitted to the ultrasound probe 2d from a transmission unit 12, reception signals transmitted from the ultrasound probe 2d to a reception unit 13 and control signals transmitted from a control unit 15 to the amplitude adjustment units 14d are conveyed through a cable 22. Except for that, the ultrasound diagnostic apparatus Sd of each of the fifth embodiment and the sixth embodiment is the same as that of the ultrasound diagnostic apparatus S of the first embodiment. The same reference numbers are given to the same components, and description thereof is omitted herein.

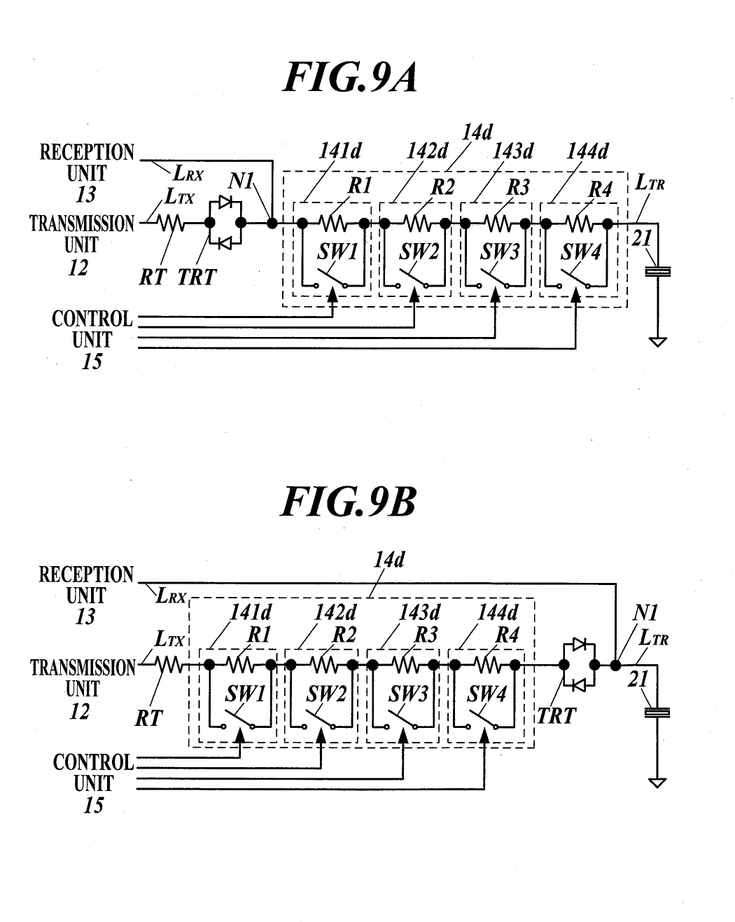

FIG. 9A shows a circuit configuration relating to transmission of a pulse signal and reception of a reception signal in the ultrasound diagnostic apparatus Sd of the fifth embodiment. FIG. 9B shows a circuit configuration relating to transmission of a pulse signal and reception of a reception signal in the ultrasound diagnostic apparatus Sd of the sixth embodiment.

In the ultrasound diagnostic apparatus Sd of the fifth embodiment, as shown in FIG. 9A, an amplitude adjustment unit 14d is disposed on a common line L_{TR}. In the amplitude adjustment unit 14d, weighting units 141d to 144d are connected in series. In the weighting unit 141d, a resistor R1 and a switch SW1 are connected in parallel. That is, in the weighting unit 141d, when the switch SW1 is ON, the ends of the resistor R1 short-circuit, and a pulse signal and a reception signal bypass the resistor R1. On the other hand, when the switch SW1 is OFF, the pulse signal and the reception signal pass through the resistor R1. Similarly, in the weighting unit 142d, a resistor R2 and a switch SW2 are connected in parallel; in the weighting unit 143d, a resistor R3 and a switch SW3 are connected in parallel; and in the weighting unit 144d, a resistor R4 and a switch SW4 are connected in parallel. When the switches SW1 to SW4 are ON and accordingly a signal bypasses the resistors R1 to R4, the load of the whole amplitude adjustment unit 14d (a partial voltage applied to the whole amplitude adjustment unit 14d) decreases, and in relation to it, a voltage applied to a transducer 21 increases. On the other hand, when the switches SW1 to SW4 are OFF and accordingly a signal passes through the resistors R1 to R4, the load of the whole amplitude adjustment unit 14d (the partial voltage applied to the whole amplitude adjustment unit 14d) increases, and in relation to it, the voltage applied to the transducer 21 decreases.

In this case, the node N1 may be disposed in a main body 1d, and the common line L_{TR} may be threaded through the cable 22.

In the ultrasound diagnostic apparatus Sd of the sixth embodiment, as shown in FIG. 9B, an amplitude adjustment unit 14d is disposed on a transmission line L_{TX}, which is for a pulse signal output from a transmission unit 12, between internal resistance RT of a pulser and a T/R switch TRT. That is, in the ultrasound diagnostic apparatus Sd of the sixth embodiment, as with the ultrasound diagnostic apparatus Sd of the fifth embodiment, resistors R1 to R4 of weighing units 141d to 144d and a transducer 21 are connected in series. Hence, the amplitude of ultrasound to be transmitted from the transducer 21 is appropriately controlled by changing a partial voltage applied to the transducer 21 by a combination of ON and/or OFF of switches SW1 to SW4 based on control signals from a control unit 15.

As described above, in the ultrasound diagnostic apparatus Sd of each of the fifth embodiment and the sixth embodiment, the amplitude adjustment units 14d are disposed in the ultrasound probe 2d. In each amplitude adjustment unit 14d, the resistors R1 to R4 in the weighting units 141d to 144d and the transducer 21 are all electrically connected in series. Consequently, unnecessary power consumption in amplitude adjustment in the amplitude adjustment units 14d can be prevented. Further, the resistance values of the resistors R1 to R4 of each amplitude adjustment unit 14d and the resistance value of each transducer 21 can be set together in forming the ultrasound probe 2d. Hence, even when there is a variation in the resistance values of transducers 21 according to ultrasound probes 2b, an appropriate output profile of amplitudes of ultrasound can be set for each ultrasound probe 2d which is connected to the main body 1d.

The amplitude adjustment units 14d may be disposed at any point of the cable 22 (a connector thereof included) too. Even when the amplitude adjustment units 14d are disposed on the cable 22, the resistance value of each transducer 21 and the resistance values of the resistors R1 to R4 can be set together. Hence, an appropriate output profile of amplitudes of ultrasound can be set for each ultrasound probe 2b.

### Seventh Embodiment and Eighth Embodiment

Next, ultrasound diagnostic apparatuses according to a seventh embodiment and an eighth embodiment of the present invention are described.

An ultrasound diagnostic apparatus S of the seventh embodiment is the same as the ultrasound diagnostic apparatus S of the first embodiment shown in FIG. 2 except that the amplitude adjustment units 14 are replaced by amplitude adjustment units 14e. The same reference numbers are given to the same components, and description thereof is omitted herein.

An ultrasound diagnostic apparatus Sa of the eighth embodiment is the same as the ultrasound diagnostic apparatus Sa of the second embodiment shown in FIG. 4 except that the amplitude adjustment units 14a are replaced by amplitude adjustment units 14e. The same reference numbers are given to the same components, and description thereof is omitted herein.

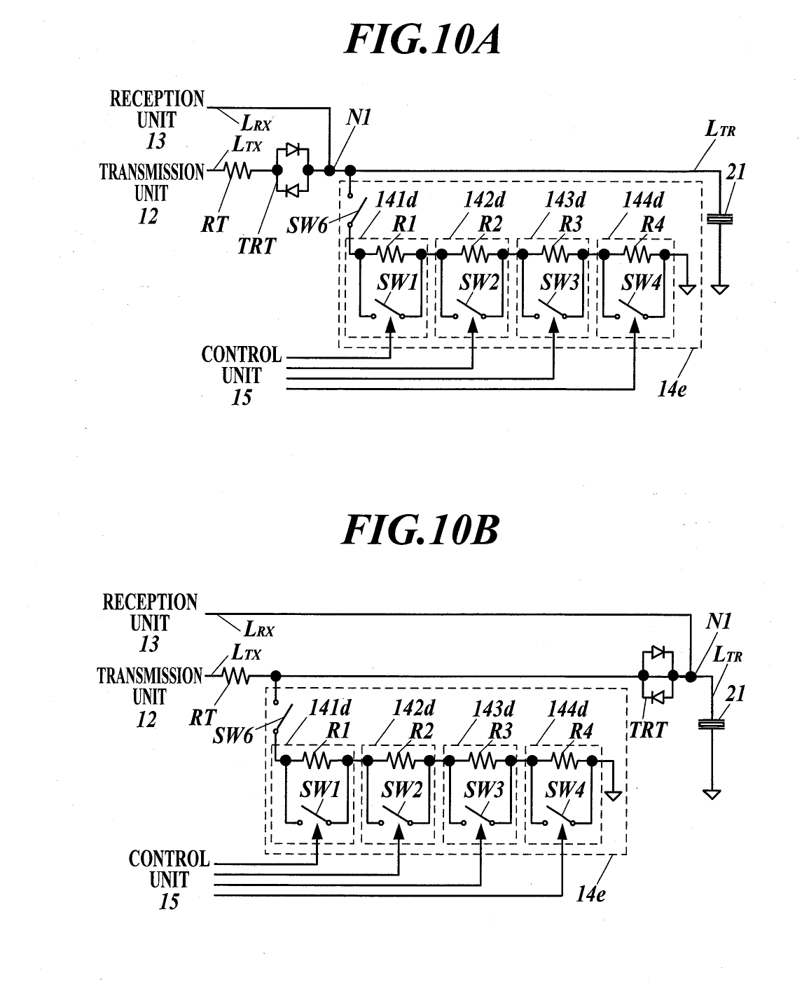

FIG. 10A shows a circuit configuration relating to transmission of a pulse signal and reception of a reception signal in the ultrasound diagnostic apparatus S of the seventh embodiment. FIG. 10B shows a circuit configuration relating to transmission of a pulse signal and reception of a reception signal in the ultrasound diagnostic apparatus Sa of the eighth embodiment.

In the ultrasound diagnostic apparatus S of the seventh embodiment, as shown in FIG. 10A, an amplitude adjustment unit 14e is connected to a transducer 21 in parallel. In the amplitude adjustment unit 14e, weighting units 141d to 144d are connected to each other in such a way that resistors R1 to R4 respectively included in the weighting units 141d to 144d are connected in series. One end of the weighting unit 141d is connected to a common line L_{TR} through a switch SW6, and one end of the weighting unit 144d is grounded.

The switch SW6 is made to be OFF on the basis of control signals from a control unit 15 when ultrasound is received so as to prevent a reception signal from flowing to the amplitude adjustment unit 14e. The switch SW6 is also made to be OFF when a voltage pulse as a pulse signal is transmitted to the transducer 21 without using the amplitude adjustment unit 14e. If there is no expectation that ultrasound is transmitted without using the amplitude adjustment unit 14e, the amplitude adjustment unit 14e may be provided with a diode switch described above instead of the switch SW6.

In the ultrasound diagnostic apparatus Sa of the eighth embodiment, as shown in FIG. 10B, an amplitude adjustment unit 14e is disposed on a transmission line L_{TX} between internal resistance RT of a pulser and a T/R switch TRT. The amplitude adjustment unit 14e is connected to a transducer 21 in parallel.

In the ultrasound diagnostic apparatus Sa of the eighth embodiment, no reception signal flows to the amplitude adjustment unit 14e owing to the T/R switch TRT, and hence ON/OFF of the switch SW6 is controlled by a control unit 15 on the basis of whether or not the amplitude adjustment unit 14e is used to transmit a pulse signal to the transducer 21. If there is no expectation that a pulse signal is transmitted to the transducer 21 without using the amplitude adjustment unit 14e, the switch SW6 may be omitted.

As described above, in each of the ultrasound diagnostic apparatus S of the seventh embodiment and the ultrasound diagnostic apparatus Sa of the eighth embodiment, in each amplitude adjustment unit 14e electrically connected to a transducer 21 in parallel, four resistors R1 to R4 are connected in series, and the switches SW1 to SW4 can provide bypasses around the resistors R1 to R4, respectively. Consequently, a partial voltage for the amplitude of ultrasound to be transmitted can be applied to the amplitude adjustment unit 14e and the transducer 21. As described above, the resistors R1 to R4 electrically connected to the transducer 21 in parallel can be connected in series instead of in parallel. With either of the configurations of these embodiments, as with the other embodiments, the amplitudes of ultrasound to be transmitted from the transducers 21 can be appropriately weighted with a simple configuration which does not to increase the circuit size and with a simple action.

### Ninth Embodiment and Tenth Embodiment

Next, ultrasound diagnostic apparatuses according to a ninth embodiment and a tenth embodiment of the present invention are described.

An ultrasound diagnostic apparatus S of the ninth embodiment is the same as the ultrasound diagnostic apparatus S of the first embodiment except that the amplitude adjustment units 14 are replaced by amplitude adjustment units 14f. An ultrasound diagnostic apparatus S of the tenth embodiment is the same as the ultrasound diagnostic apparatus S of the first embodiment except that the amplitude adjustment units 14 are replaced by amplitude adjustment units 14g. The same reference numbers are given to the same components, and description thereof is omitted herein.

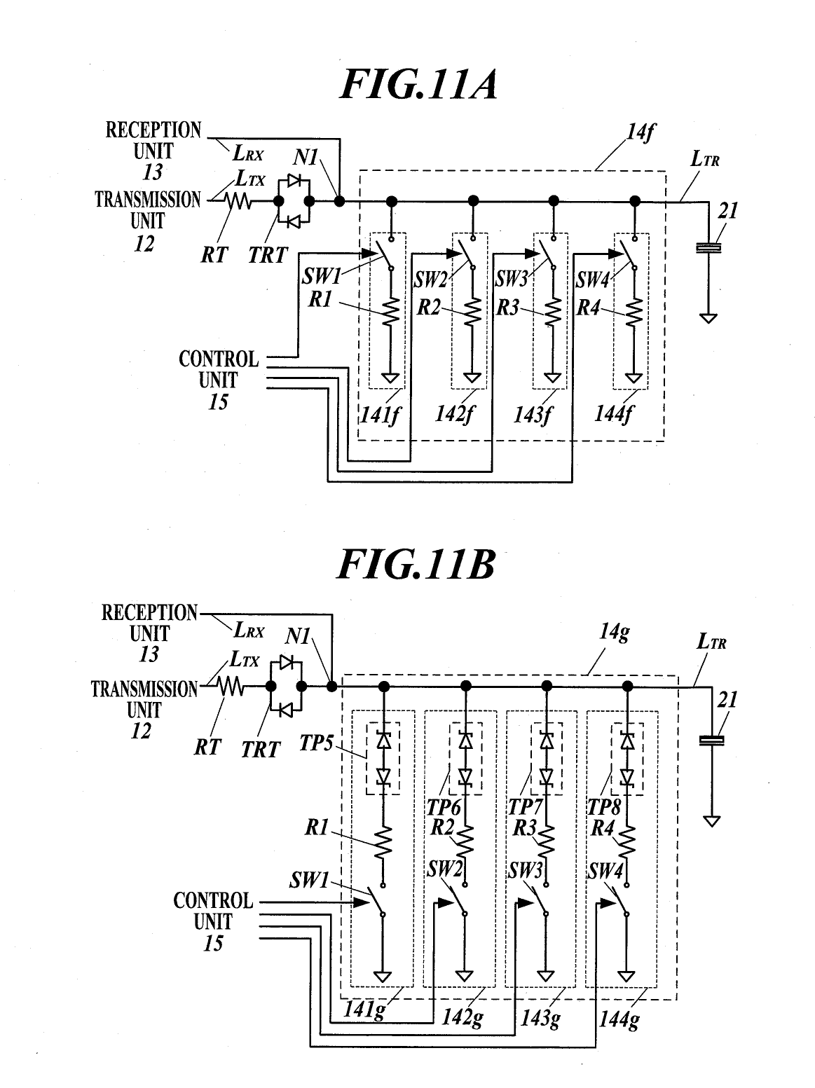

FIG. 11A shows a circuit configuration relating to transmission of a pulse signal and reception of a reception signal in the ultrasound diagnostic apparatus S of the ninth embodiment. FIG. 11B shows a circuit configuration relating to transmission of a pulse signal and reception of a reception signal in the ultrasound diagnostic apparatus S of the tenth embodiment.

As shown in FIG. 11A, in an amplitude adjustment unit 14f of the ultrasound diagnostic apparatus S of the ninth embodiment, four weighting units 141f to 144f are electrically connected to a transducer 21 in parallel between a node N1 and a ground level. In the weighting unit 141f, a switch SW1 is disposed on the node N1 side, a resistor R1 is disposed on the ground side, and the switch SW1 and the resistor R1 are connected in series. Similarly, in the weighting unit 142f, a switch SW2 is disposed on the node N1 side, a resistor R2 is disposed on the ground side, and the switch SW2 and the resistor R2 are connected in series; in the weighting unit 143f, a switch SW3 is disposed on the node N1 side, a resistor R3 is disposed on the ground side, and the switch SW3 and the resistor R3 are connected in series; and in the weighting unit 144f, a switch SW4 is disposed on the node N1 side, a resistor R4 is disposed on the ground side, and the switch SW4 and the resistor R4 are connected in series. The position of each of the switches SW1 to SW4 and the position of each of the resistors R1 to R4 may be reversed to be the same as those in an amplitude adjustment unit 14a.

In the amplitude adjustment unit 14f of the ultrasound diagnostic apparatus S of the ninth embodiment, when ultrasound is transmitted, a voltage of the node N1, namely, a voltage applied to the transducer 21, is appropriately changed by a combination of ON and/or OFF of the switches SW1 to SW4 based on control signals from a control unit 15. On the other hand, when ultrasound is received, the whole reception signal from the transducer 21 is input to a reception unit 13 by making all the switches SW1 to SW4 OFF based on control signals from the control unit 15.

As described above, in the ultrasound diagnostic apparatus S of the ninth embodiment, on the contrary to the ultrasound diagnostic apparatus S of the first embodiment, ON/OFF of each of the switches SW1 to SW4 is controlled at high speed at the time when transmission and reception of ultrasound are switched. Consequently, signal paths for the pulse signal and the reception signal can be appropriately controlled with no diode switch, and hence the amplitude adjustment unit 14f can be smaller.

In an amplitude adjustment unit 14g of the ultrasound diagnostic apparatus S of the tenth embodiment, as shown in FIG. 11B, four weighting units 141g to 144g are electrically connected to a transducer 21 in parallel between a node N1 and a ground level. The weighting units 141g to 144g are the same as the weighting units 141 to 144 of the first embodiment except that the diode switches TP1 to TP4 are respectively replaced by diode switches TP5 to TP8. Each of the diode switches TP5 to TP8 is composed of two zener diodes disposed in anti-series. This configuration allows a voltage signal with a voltage equal to or more than a breakdown voltage of the zener diode connected in the reverse direction to an applied voltage to pass and blocks a voltage signal with a voltage less than the breakdown voltage. Consequently, a reception signal with a small voltage amplitude does not flow to the amplitude adjustment unit 14g and is transmitted to a reception unit 13, whereas a pulse signal with a large voltage amplitude flows to the amplitude adjustment unit 14g and the voltage amplitude of the pulse signal applied to a transducer 21 is adjusted.

The breakdown voltage of the zener diode can be set to a proper value within a range of values stably settable. By setting this value to be sufficiently small to the voltage amplitude of the pulse signal and sufficiently large to the expected voltage amplitude of the reception signal, bad influence is not exerted on either the waveform of the pulse signal or the waveform of the reception signal. Further, even if a desired output voltage profile is difficult to set by using commercial resistors R1 to R4 in the weighting units 141g to 144g, an output voltage can be adjusted with the breakdown voltage.

As described above, in the ultrasound diagnostic apparatus S of the tenth embodiment, the weighting units 141g to 144g include the diode switches TP5 to TP8, respectively. Consequently, the pulse signal which should be allowed to pass through the weighting units 141g to 144g and the reception signal which should not be allowed to pass through the weighting units 141g to 144g can be easily separated from each other with a simple configuration which does not to increase the circuit size and without ON/OFF control of the switches SW1 to SW4 performed at the time when transmission and reception of ultrasound are switched.

### Eleventh Embodiment

Next, an ultrasound diagnostic apparatus according to an eleventh embodiment of the present invention is described.

An ultrasound diagnostic apparatus S of the eleventh embodiment is the same as the ultrasound diagnostic apparatus S of the first embodiment except that, in the eleventh embodiment, amplitude adjustment units 14 each are connected to one of a plurality of connectable transducers 21. The same reference numbers are given to the same components, and description thereof is omitted herein.

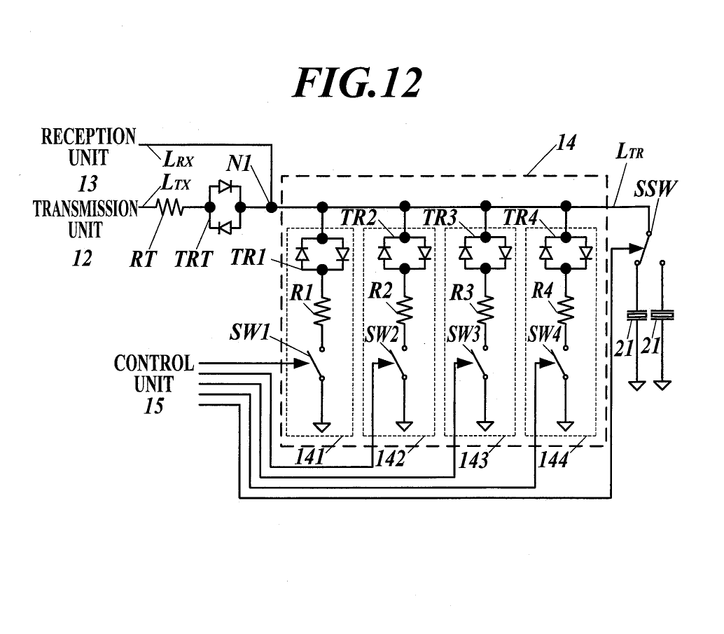

FIG. 12 shows a circuit configuration relating to transmission of a pulse signal and reception of a reception signal in the ultrasound diagnostic apparatus S of the eleventh embodiment.

In the ultrasound diagnostic apparatus S of the eleventh embodiment, an amplitude adjustment unit 14 is connected to one of a plurality of connectable transducers 21 through a switch SSW. In the eleventh embodiment, two transducers 21 are shown as an example of the transducers 21 connectable to one amplitude adjustment unit 14. However, the number of connectable transducers 21 may be more than two. Transducers 21 connectable to one amplitude adjustment unit 14 and transducers 21 connectable to another amplitude adjustment unit 14 are not exclusive thereto, and each transducer 21 can be connected to one of a plurality of amplitude adjustment units 14. For example, the switches SSW are switched in a state in which ON/OFF states of switches SW1 to SW4 of the amplitude adjustment units 14 are fixed so as to shift the transducers 21 connected to the amplitude adjustment units 14 by one transducer 21, so that the direction of ultrasound to be transmitted (transmission ultrasound) from an ultrasound probe 2 can be changed with a transmission ultrasound profile determined by the amplitude adjustment units 14 kept, namely, scanning can be performed.

In this case, the number of amplitude adjustment units 14 can be less than the number of transducers 21, and a transducer(s) 21 which does not transmit/receive ultrasound is not connected to any of the amplitude adjustment units 14.

According to this kind of ultrasound diagnostic apparatus S, the number of situations where ON/OFF of not the plurality of switches SW1 to SW4 but only one switch SSW in each amplitude adjustment unit 14 needs to be changed increases, so that, as a whole, the output amount of control signals from a control unit 15 can be reduced and control can be simple and easy. In this case, switching frequencies of the switches SW1 to SW4 decrease, so that, instead of FETs, other switches having high voltage resistance, such as mechanical relay switches, may be used as the switches SW1 to SW4.

As described above, the ultrasound diagnostic apparatus S of the eleventh embodiment includes the switches SSW each of which sets a connection relationship between an amplitude adjustment unit 14 and transducers 21. With this configuration, each amplitude adjustment unit 14 is connected, on the basis of a control signal from the control unit 15, to a transducer 21 which outputs ultrasound with an amplitude set by the amplitude adjustment unit 14. Consequently, the switching frequencies of many switches SW1 to SW4 included in the amplitude adjustment units 14 can be reduced and a processing load can be prevented from increasing.

Further, as the switches (SW1 to SW4) having low switching frequencies, mechanical relay switches can be used, so that electrical continuity can be physically broken. Consequently, the resistance against a high applied voltage can be obtained more certainly.

The present invention is not limited to the above-described embodiments and hence can be variously modified.

For example, in the embodiments, all the resistors R1 to R4 in each amplitude adjustment unit are connected in parallel or in series. However, if a set of resistors is formed to use on the basis of a relationship between a desired profile and an easily-obtainable resistance value, it is possible that the resistors of the set are connected in series and controlled with one switch, and the other resistor(s) is connected to the resistors of the set in parallel.

Further, in the embodiments, the diode switches using the forward voltage characteristics of diodes or the diode switches using the zener breakdown voltage of the reverse voltage are used. However, these types of diode switches may coexist depending on the resistors connected to the switches in series.

Further, in the embodiments, the main body and the ultrasound prove are combined to configure the ultrasound diagnostic apparatus S, Sa or Sd. However, in the case where the amplitude adjustments (any of 14 to 14g) are disposed inside the main body, the ultrasound diagnostic apparatus of the present invention may be composed of the main body only, and this ultrasound diagnostic apparatus can be used by being connected to an external ultrasound probe attachable to the main body.

Further, only the amplitude adjustment units 14d of the ultrasound diagnostic apparatus Sd of each of the fifth embodiment and the sixth embodiment are disposed inside the ultrasound prove 2d. However, the amplitude adjustment units of the ultrasound diagnostic apparatus S or Sa of each of the other embodiments may also be disposed inside the ultrasound probe 2d (or at any point of the cable 22 including the connector). Alternatively, the amplitude adjustment units 14d may also be disposed inside the main body 1 or 1a.

Further, in the embodiments, the bipolar rectangular pulses are applied to the transducers 21. However, the waveform of the pulse signals can be set appropriately. It is preferable that amplitude adjustment units suitable for the waveform be selected from the amplitude adjustment units 14 to 14g.

The specific details such as the configurations and the circuits described in the above-described embodiments can be appropriately modified without departing from the scope of the present invention.

This application is based upon and claims the benefit of priority under 35 USC 119 of Japanese Patent Application No. 2013-041379 filed on Mar. 4, 2013, the entire disclosure of which, including the description, claims, drawings and abstract, is incorporated herein by reference in its entirety.

What is claimed is:

1. An ultrasound diagnostic apparatus which outputs a pulse signal to each of a plurality of transducers to make each of the transducers transmit ultrasound and obtains a reception signal based on ultrasound received by each of the transducers, the ultrasound diagnostic apparatus comprising:
a signal generation unit which generates the pulse signal with a predetermined voltage amplitude;
a plurality of amplitude adjustment units which individually change the voltage amplitude of the pulse signal so as to output pulse signals with individual voltage amplitudes to the respective transducers; and
a control unit which controls change amounts of the voltage amplitude made by the amplitude adjustment units, wherein
each of the amplitude adjustment units includes a plurality of resistive elements and a plurality of switching units which change a conductive state of the respective resistive elements,
the control unit outputs a control signal to set a combination of ON and/or OFF of the switching units to change a load of the amplitude adjustment unit so as to apply a voltage for an amplitude of the ultrasound, which is transmitted from each of the transducers, to the transducer.

2. The ultrasound diagnostic apparatus according to claim 1, wherein the resistive elements in the amplitude adjustment unit are electrically connected to the transducer in parallel.

3. The ultrasound diagnostic apparatus according to claim 2, wherein two diode elements disposed in antiparallel are electrically connected to each of the resistive elements and each of the switching units in series in the amplitude adjustment unit.

4. The ultrasound diagnostic apparatus according to claim 1 further comprising an ultrasound probe including the transducers and a connection unit to be connected with a main body of the ultrasound diagnostic apparatus, wherein
the amplitude adjustment units are disposed in the ultrasound probe.

5. The ultrasound diagnostic apparatus according to claim 2 further comprising an ultrasound probe including the transducers and a connection unit to be connected with a main body of the ultrasound diagnostic apparatus, wherein
the amplitude adjustment units are disposed in the ultrasound probe.

6. The ultrasound diagnostic apparatus according to claim 3 further comprising an ultrasound probe including the transducers and a connection unit to be connected with a main body of the ultrasound diagnostic apparatus, wherein
the amplitude adjustment units are disposed in the ultrasound probe.

7. The ultrasound diagnostic apparatus according to claim 4, wherein the resistive elements in each of the amplitude adjustment units are electrically connected to each of the transducers in series.

8. The ultrasound diagnostic apparatus according to claim 5, wherein the resistive elements in each of the amplitude adjustment units are electrically connected to each of the transducers in series.

9. The ultrasound diagnostic apparatus according to claim 6, wherein the resistive elements in each of the amplitude adjustment units are electrically connected to each of the transducers in series.

10. The ultrasound diagnostic apparatus according to claim 1 further comprising a signal processing unit which processes the reception signal, wherein with respect to each of the amplitude adjustment units and each of the transducers,
a reception line to convey the reception signal to the signal processing unit and a transmission line to convey the pulse signal from the signal generation unit are connected at a predetermined node to a common line connected to the transducer,
the amplitude adjustment unit is disposed on the transmission line, and
between the node and the amplitude adjustment unit, a selective passing unit which selectively allows the pulse signal to pass is disposed.

11. The ultrasound diagnostic apparatus according to claim 2 further comprising a signal processing unit which processes the reception signal, wherein with respect to each of the amplitude adjustment units and each of the transducers,
a reception line to convey the reception signal to the signal processing unit and a transmission line to convey the pulse signal from the signal generation unit are connected at a predetermined node to a common line connected to the transducer,
the amplitude adjustment unit is disposed on the transmission line, and
between the node and the amplitude adjustment unit, a selective passing unit which selectively allows the pulse signal to pass is disposed.

12. The ultrasound diagnostic apparatus according to claim 3 further comprising a signal processing unit which processes the reception signal, wherein with respect to each of the amplitude adjustment units and each of the transducers,
a reception line to convey the reception signal to the signal processing unit and a transmission line to convey the pulse signal from the signal generation unit are connected at a predetermined node to a common line connected to the transducer,
the amplitude adjustment unit is disposed on the transmission line, and
between the node and the amplitude adjustment unit, a selective passing unit which selectively allows the pulse signal to pass is disposed.

13. The ultrasound diagnostic apparatus according to claim 1, wherein the switching units each are an analog switch or a mechanical relay switch.

14. The ultrasound diagnostic apparatus according to claim 1, wherein resistance values of the resistive elements in the amplitude adjustment unit are all different from each other.
